(19) United States
(12) Patent Application Publication
(10) Pub. No.: US 2025/0149188 A1
(43) Pub. Date: May 8, 2025

(54) AUTOMATIC GENERATION OF HEALTHCARE APPLICATION FROM HEALTHCARE PROTOCOL AND MEDICAL INFORMATION SOURCES

(71) Applicant: GoForward, Inc., San Francisco, CA (US)

(72) Inventors: Adrian Aoun, San Francisco, CA (US); Eugene Leonard Huang, San Francisco, CA (US); Ilya Abyzov, Larkspur, CA (US)

(21) Appl. No.: 18/915,391

(22) Filed: Oct. 15, 2024

Related U.S. Application Data
(60) Provisional application No. 63/596,595, filed on Nov. 6, 2023.

(51) Int. Cl. G16H 70/20; G16H 50/20

(52) U.S. Cl. CPC G16H 70/20; G16H 50/20

(57) ABSTRACT

Embodiments relate to implementing a healthcare protocol on a medical station by generating an application that incorporates the healthcare protocol. The healthcare protocol may be generated by collecting materials from sources and processing them into code for execution on the medical station by mapping portions of the healthcare protocol to functionalities of the medical station. For this purpose, the healthcare protocol may be represented as a decision tree tree or a Boolean logic. The generated code and related content may be packaged into the application. The application is sent to the medical station and deployed to implement the healthcare protocol on the medical station.

### CROSS-REFERENCE TO RELATED APPLICATION

This application claims priority under 35 U.S.C. § 119(e) to U.S. Patent Application No. 63/596,595, filed on Nov. 6, 2023, which is incorporated by reference herein in its entirety.

### BACKGROUND

Embodiments relate to using healthcare protocols to generate software applications, and more specifically to automatically generate software applications for use in modular medical stations using healthcare protocols.

A healthcare protocol refers to a standardized set of guidelines, rules, or procedures that outline recommended steps and actions to be followed in the diagnosis, treatment, management, or prevention of various medical conditions or situations. Such healthcare protocols are generally used to ensure consistent and a level of care across different healthcare settings and different healthcare professionals. The healthcare protocols may apply to various areas of healthcare and wellness including clinical diagnosis and treatment, addressing of emergency medical situations, care process for certain medical conditions, managing of chronic conditions such as diabetes or hypertension, conducting stress management intervention, assisting meditation, prescribing fitness programs, and recommending preventive measures.

These healthcare protocols evolve over time due to new research, technology, clinical experiences, patient preferences, and changing health challenges. Emerging evidence, medical guidelines, and advances in tools and treatments may drive updates to the healthcare protocols or creation of new healthcare protocols. Hence, regular reviews and updates may be performed to ensure that the healthcare protocols reflect the result of recent knowledge and available diagnostic/treatment options. These reviews and updates to the healthcare protocols may involve a significant amount of time and resources of the health professionals and health systems.

### SUMMARY

Embodiments relate to generating an application implementing a healthcare protocol for execution by a medical station. The healthcare protocol is associated with diagnosis or treatment of a user or preventing health issues of the user accessing the medical station. Instructions are sent to a language model to generate the healthcare protocol. The healthcare protocol is received from the language model after sending the instructions to the language model. Mapping between segments in the healthcare protocol and functionalities of the medical station is determined. Each of the segments includes one or more nodes of the healthcare protocol. Logic corresponding to sequence of the segments and the mapping is generated. An application incorporating the logic is generated for execution by the medical station.

In one or more embodiments, the healthcare protocol is represented as a decision tree or a Boolean logic including the one or more nodes.

In one or more embodiments, information on tools, sensors and plug-in components on the medical station is stored. The functionalities mapped to the segments are associated with the tools, sensors and plug-in components.

In one or more embodiments, the healthcare protocol indicates one or more actions to be taken by at least one of the user or the medical station according to determination at the segments.

In one or more embodiments, materials on the healthcare protocols are collected from a plurality of sources. The collected materials are processed and fed to the language model to fine-tune or perform training of the language model.

In one or more embodiments, the collected materials are filtered before feeding to the language model.

In one or more embodiments, content is generated using at least one of graphics engine, a speech synthesizer and a generative artificial intelligence. The application further incorporates the generated content.

In one or more embodiments, the logic is compiled into code executable by the medical station. The content is linked to the executable code. The executable code and the content are packaged into the application.

In one or more embodiments, the application is sent to the medical station via a network for execution by the medical station.

### BRIEF DESCRIPTION OF THE DRAWINGS

FIG. 1 is a block diagram illustrating the overall network environment including medical stations, according to one embodiment.

FIG. 2 is a block diagram illustrating components of a protocol management device, according to one embodiment.

FIG. 3A is a block diagram illustrating components of a backend server, according to one embodiment.

FIG. 3B is a block diagram illustrating the architecture of a language model in a language model server, according to one embodiment.

FIG. 4A is a block diagram illustrating components of a medical station, according to one embodiment.

FIG. 4B is a block diagram of a user device, according to one embodiment.

FIG. 5 is a conceptual diagram of a healthcare protocol and its mapping to functionality of the medical station, according to one embodiment.

FIG. 6 is a flowchart illustrating operations at the protocol management device, according to one embodiment.

### DETAILED DESCRIPTION OF EMBODIMENTS

Embodiments are described herein with reference to the accompanying drawings. Principles disclosed herein may, however, be embodied in many different forms and should not be construed as being limited to the embodiments set forth herein. In the description, details of well-known features and techniques may be omitted to avoid unnecessarily obscuring the features of the embodiments. In the drawings, like reference numerals in the drawings denote like elements. The shape, size and regions, and the like, of the drawing may be exaggerated for clarity.

Embodiments relate to implementing a healthcare protocol on a medical station by generating an application that incorporates the healthcare protocol. The healthcare protocol may be generated by collecting materials from sources and processing them into code for execution on the medical station by mapping portions of the healthcare protocol to functionalities of the medical station. For this purpose, the healthcare protocol may be represented as a decision tree or a Boolean logic. The generated code and related content may be packaged into the application. The application is sent to the medical station and deployed to implement the healthcare protocol on the medical station.

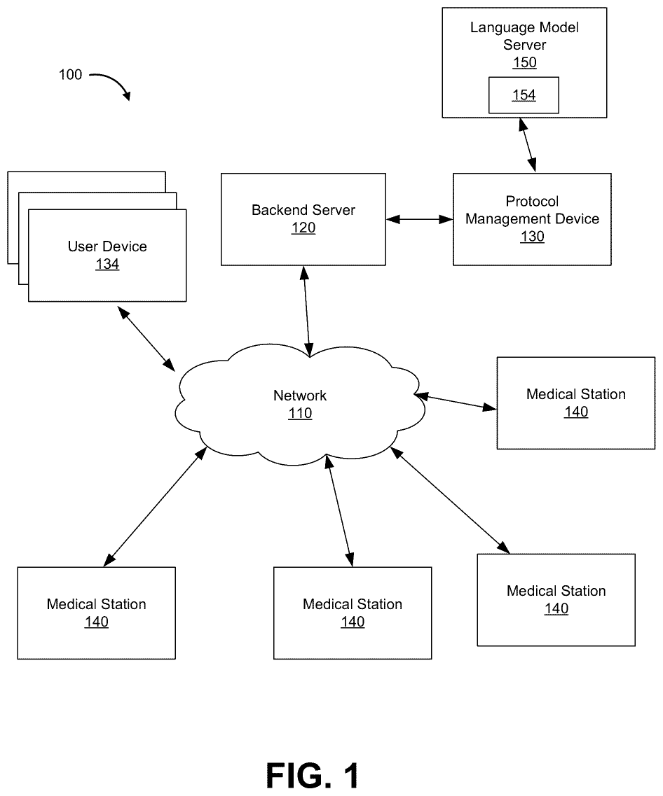

FIG. 1 is a block diagram illustrating overall network environment 100 including medical stations 140, according to one embodiment. Network environment may include, among other components, a backend server 120, a protocol management device 130, user devices 134, medical stations 140 and language model server 150. Some or all of these components communicate over network 110. Although protocol management device 130 is illustrated in FIG. 1 as communicating directly with backend server 120, protocol management device 130 may communicate with backend server 120 and/or medical stations 140 via network 110.

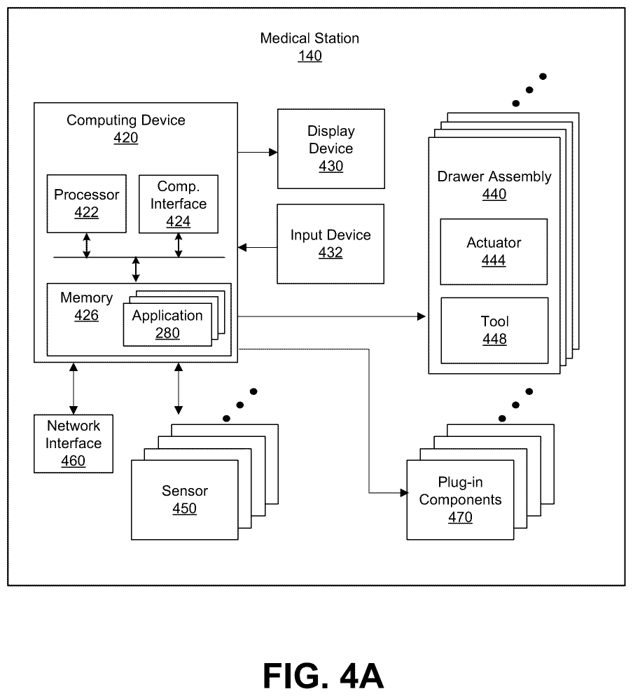

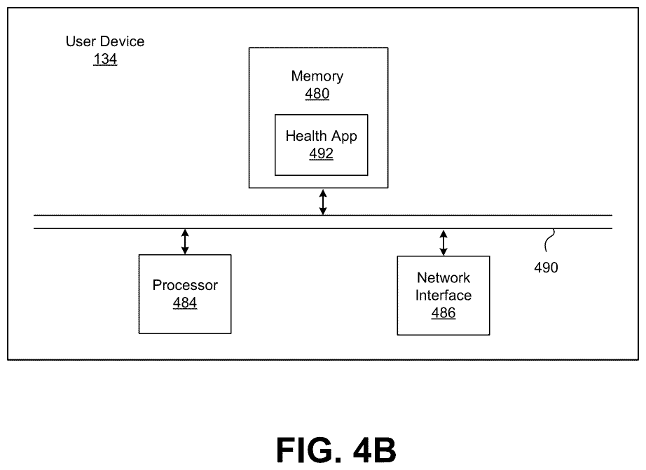

A medical station described herein is a prefabricated modular structure equipped with sensors and medical tools to provide medical services to patients or visitors. Medical station 140 may have a predetermined dimension, such as 10 feet by 15 feet or any other suitable dimensions, that can be installed inside or outside a building. An example structure of medical station 140 is described below in detail with reference to FIG. 4A. Medical station 140 communicates with backend server 120 to send and receive login information of the patient or visitor accessing medical station 140, sends and receives information associated with applications launched at medical station 140, sends information on inventories of medical tools in medical station 140, and receives applications 280 for execution.

Due to their relatively small size and the ease of installation, medical stations 140 may be beneficially installed at diverse geographical locations at relatively low cost. Medical stations 140 may be mass produced at a factory and have forms and structures adapted for deployment with reduced construction fees. A patient or a visitor may visit one of many sites installed with medical stations to receive medical services. In one or more embodiments, medical stations 140 may be operable shortly after plugging them into power outlets and setting up wireless or wired communication. Furthermore, medical stations 140 can be relocated to another site with ease, depending on, for example, changing needs at different sites. In some embodiments, the medical stations 140 may be deployed outdoors, may be mobile or otherwise movable (e.g., via built-in wheels or other transportation infrastructure), may be self-leveling (e.g., via one or more position or orientation adjustment mechanisms), and/or may be pre-assembled or pre-manufactured for on-site assembly.

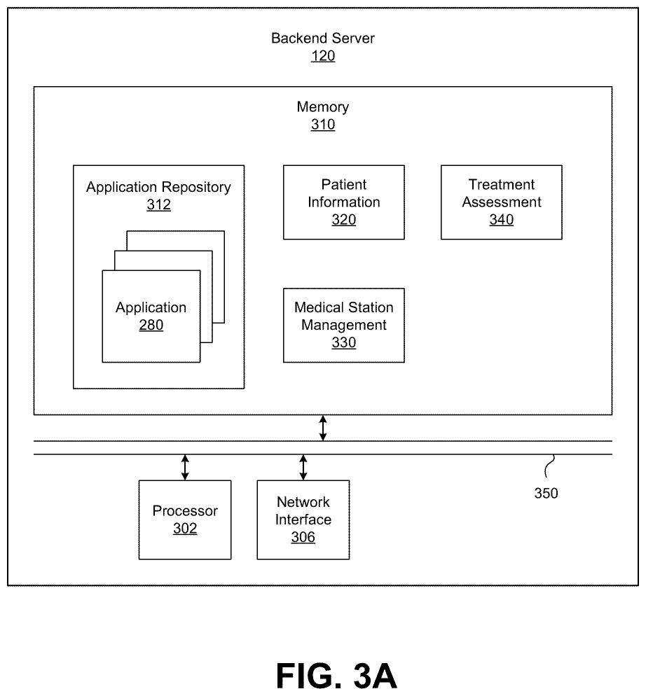

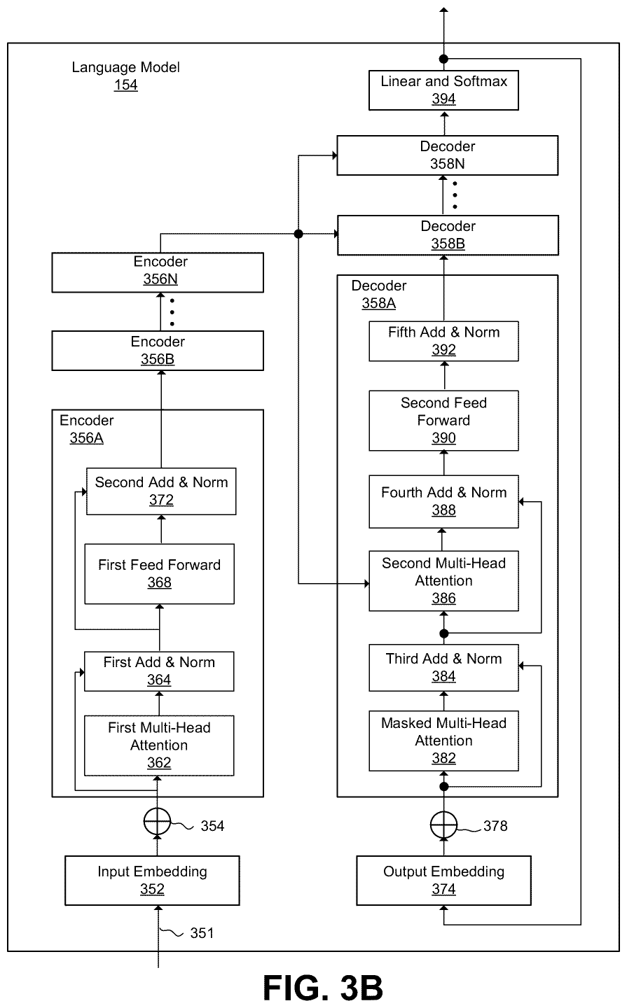

Backend server 120 is a computing device that facilitates or supports medical station 140 to provide medical services to its patients or visitors. Backend server 120 may store various applications 280 from various sources, including protocol management device 130, and may send the applications 280 to medical stations 140 for deployment. Backend server 120 may also evaluate performance of various applications 280 by collecting information received from medical stations 140, and update/discard applications 280 upon performance and/or demand. The components and functions of backend server 120 are described below in detail with reference to FIG. 3A.

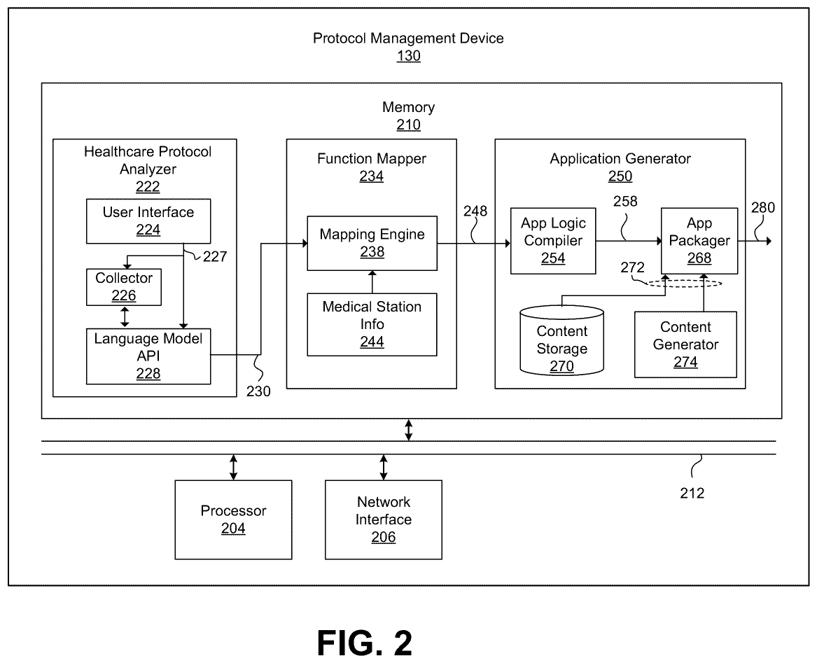

Protocol management device 130 is a computing device for collecting materials and generating healthcare protocols from the collected materials. The generated healthcare protocols may be in the form of intermediate data from the healthcare protocols, and which is in turn converted into software applications 280 for deployment at medical stations 140. For this purpose, protocol management device 130 may collect materials (e.g., medical journals, medical articles, medical database, websites and medical books) on a certain field of healthcare (e.g., cardiology), use artificial intelligence (AI) language models (e.g., large language models) in language model server 150, and automatically generate software applications 280 suitable for execution on medical stations 140, as described below in detail with reference to FIG. 2.

Although only a single protocol management device 130 is illustrated in FIG. 1, in practice, there may be many protocol management devices where each of the protocol management devices is operated by different medical service providers. Protocol management device 130 may be operated by the same entity that operates medical stations 140. Alternatively, each service developer device 130 may be managed and operated by an entity different from the entity that operates medical stations 140. By automating the process of generating applications 280 executable on medical stations 140, medical service providers may easily develop their own applications without detailed technical information on medical stations 140.

User devices 134 are computing devices accessed by patients or visitors to take various actions associated with healthcare services. A user device 134 may be embodied as a stationary or portable computing device such as personal computers, smartphones, consoles, smart TVs, and set-top boxes. Each of the user devices 134 may be associated with a single patient/visitor or multiple patients/visitors. User devices 134 may perform various operations such as launching and executing health apps that enable the patents/visitors to reserve or access medical stations 140, take actions associated with medical records (e.g., create, update or delete medical records), receive various notifications from backend server 120 or medical stations 140. An example user device 134 is described below in detail with reference to FIG. 4B.

Language model server 150 is a computing device including an artificial intelligence (AI) language model 154 that is trained to process and generate information in the form of text or other formats. Language model server 150 may be dedicated to providing a specific service or generic services depending on the language model used, the training data used, and availability of application program interface (API) to access language model. Language model server 150 may use various types of AI language models, as described below in detail with reference to FIG. 3B. Although language model server 150 is illustrated in FIG. 1 as being connected directly to protocol management device 130, language model server 150 may be connected to protocol management device 130 and provide services to multiple devices and users.

Network 110 is a set of devices that enable computing devices to communicate with each other. Various communication protocols may be used by network 110 including, but not limited to, Internet Protocols, Ethernet and wireless network protocols such as cellular standards. In one or more embodiments, network 110 includes routers, switches and servers.

The architecture of FIG. 1 is merely illustrative and various changes may be made. For example, language model server 150 may be combined with backend server 120 or protocol management device 130. Further, backend server 120 may be combined with protocol management device 130. Also, some medical stations 140 may communicate with backend server 120 via another medical station 140. AI language model in language model server 150 may also be included in protocol management device 130 instead.

FIG. 2 is a block diagram illustrating components of protocol management device 130, according to one embodiment. Protocol management device 130 may include, among other components, processor 204, memory 210, network interface 206 and bus 212 connecting these components. Protocol management device 130 may include other components not illustrated in FIG. 2.

Processor 204 reads and executes instructions stored in memory 210 to perform various operations on protocol management device 130. Although only a single processor 204 is illustrated in FIG. 2, multiple processors may be included in protocol management device 130. These processors may be provided as separate discrete chips or be included in a single chip such as in a system-on-a-chip (SoC) implementation. Processor 204 may be general-purpose or an embedded processor using any of a variety of instruction set architectures (ISAs), such as the x86, PowerPC, SPARC, RISC, ARM or MIPS ISAs, or any other suitable ISAs.

Network interface 206 is hardware or hardware in combination with firmware or software for communicating with medical stations 140 and/or backend server 120. For this purpose, network interface 206 may implement various wired or wireless protocols.

Memory 210 is a non-transitory storage medium for storing software modules. Memory 210 may include, among other software modules, healthcare protocol analyzer 222, function mapper 234 and application generator 250. Memory 210 may store other software modules not illustrated in FIG. 2. Two or more software modules in FIG. 2 may be combined into a single module or a software module in FIG. 2 may be split up into multiple software modules. Memory 210 may take the form of any type of memory structure including, for example, dynamic random access memory (DRAM), synchronous DRAM (SDRAM), double data rate (DDR, DDR2, DDR3, etc.) RAMBUS DRAM (RDRAM), static RAM (SRAM) or a combination thereof.

Healthcare protocol analyzer 222 is a software module that interfaces with an AI language model in language model server 150 to analyze materials on healthcare protocols, and generate intermediate data 230 for subsequent processing. For this purpose, healthcare protocol analyzer 222 may include, among other components, a user interface 224, a collector 226, and language model application programming interface (API) 228. Healthcare protocol analyzer 222 may include additional components not illustrated in FIG. 2.

User interface 224 enables a user to provide an input 227 specifying a topic or a field of healthcare protocol with relevant qualifiers, restrictions or expansions. For example, the user may provide a text input using user interface 224 to request collection and processing of materials on cardiovascular health. The text input may further include qualifiers or restrictions specifying that the materials be limited in scope, for example, to materials that were published within the last 2 years and/or that the materials involve the use of certain medical tools or equipment. In addition or alternative to the text input, user interface 224 may also enable the user to provide inputs using graphical user interfaces to facilitate the user to provide key words on the topic/field and restrictions/expansions. In some embodiments, input 227 may be retrieved from a database or software components instead of being provided from the user.

In response, input 227 may be provided to collector 226 and language model API 228. Collector 226 is a software component that collects, compiles and/or processes materials on healthcare protocols corresponding to input 227 from various sources. Collector 226 is embodied, for example, as a web crawler, an Internet bot or a document parsing software that collects or extracts relevant information from the Internet, database, documents, and other sources. The materials on the healthcare protocols may be available from various public and/or private repositories such as health organizations, government health departments, medical associations and societies, health institutions and hospitals, academic and research institutions, pharmaceutical companies, online medical databases and human medical professionals (e.g., doctors and nurses). Collector 226 accesses these sources to retrieve these materials according to input 227 and provides the retrieved materials to language model API 228. For this purpose, collector 226 may store IDs and passwords for accessing these sources, and include APIs or other software components for retrieving the materials from these sources. The materials on healthcare protocol may be in the form of, for example, science journals, official reports, guideline documents, websites and online resources, manual and handbooks, educational materials, clinical practice guidelines and white papers. If the materials are not in a format amenable to further processing at protocol management device 130, collector 226 may perform further operations such as optical character recognition (OCR) on the materials. Further, collector 226 may filter materials to extract a subset of materials or assign weight values to the materials using one or more criteria. The criteria may include, among others, whether the materials are consistent with other materials, whether the reliability of the materials is above a threshold, whether the materials are cited by other materials, and the nature of the materials (e.g., whether the materials are clinical study reports (CSRs) or a meta-analysis report). The materials collected by collector 226 are then fed to language model API 228 as data for fine-tuning or further training of the language model in language model server 150.

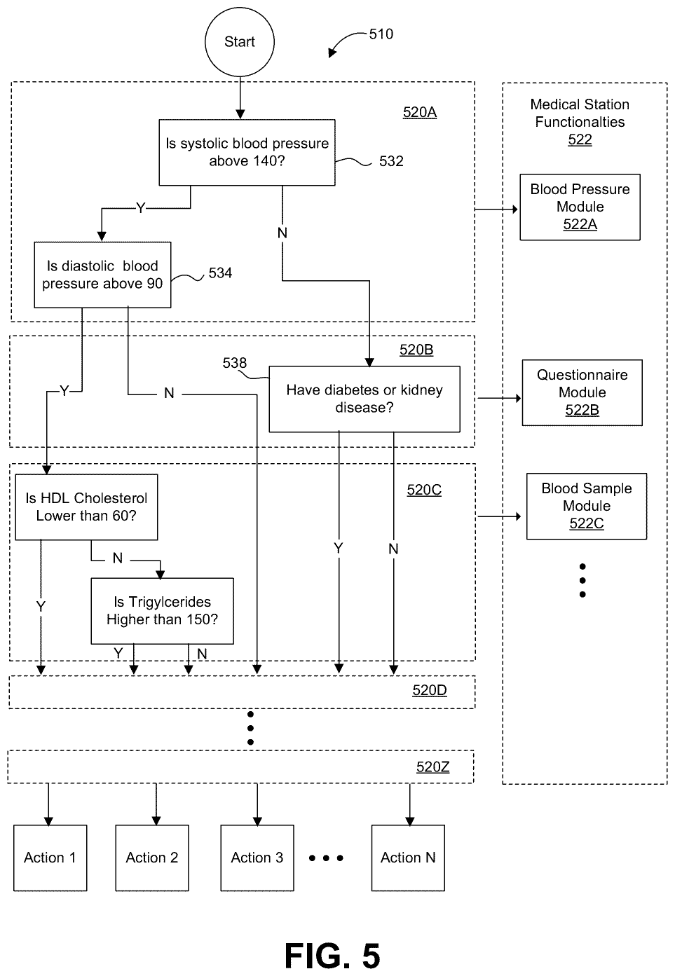

Language model API 228 is a software component that enables protocol management device 130 to interoperate with language model server 150. Language model API 228 formats and processes the materials received from collector 226 and input 227 into data and request suitable for processing by the language model in language model server 150, sends the data and request to language model server 150, and receives a healthcare protocol from language model server 150 as a response to the request. Language model API 228 may formulate the healthcare protocol received from language model server 150 into intermediate data 230 that is suitable for further processing at protocol management device 130. Intermediate data 230 may be in the form of, for example, a decision tree. An example of intermediate data 230 is described below in detail with reference to FIG. 5. Language model API 228 may also provide requests or commands to collector 226 to refine or add the collection of materials, as received from the language model or other software. In response to receiving such requests or commands, collector 226 may conduct further searches or retrieval and provide the result to language model API 228.

Function mapper 234 is a software component that receives intermediate data 230 and generates logic 248 to application generator 250. Function mapper 234 stores information 244 on medical stations. Information 244 on medical stations may indicate, for example, tools, sensors and plug-in components of medical stations that enable functionalities of the medical stations. Information 244 may also indicate the specification and details of different versions or variations in medical stations, if any.

Based on information 244 on the medical stations, mapping engine 238 maps one or more nodes of intermediate data 230 to one or more functionalities of medical station 140. A functionality of medical station 140 refers to a unit of diagnostic or treatment operation that may be performed by tools, sensors, plug-in components of a medical station or combinations thereof. Different functionalities of medical station 140 may involve different tools, sensors, plug-in components or their combinations. An example of mapping one or more nodes of intermediate data 230 to functionalities of medical stations 140 is described below in detail with reference to FIG. 5. Mapping engine 238 may be embodied as a rule engine that executes one or more rules to map the one or more nodes of the intermediate data 230 to the functionalities associated with tools, sensors, plug-in components or their combinations in medical station 140. As a result of mapping operations, mapping engine 238 generates logic 248 that embodies a healthcare protocol. Logic 248 indicates a series of functionalities of medical stations 140 that may be invoked to execute the healthcare protocol.

Application generator 250 is a software component that receives logic 248 and compiles it into an application 280 that may be executed on medical stations 140. Application generator 250 may include, among other components, application logic compiler 254, an application packager 268, content storage 270 and content generator 274. Application generator 250 may include components other than what are illustrated in FIG. 2 or omit some of these components.

Application logic compiler 254 receives logic 248 and compiles it into lines of code 258 that are applicable to medical stations 140. In one or more embodiments, a human operator may review logic 248 and/or these lines of code to confirm any issues or conflicts. Lines of code mapped to the functionalities of medical stations may be stored in advance in application logic compiler 254. Based on the stored mapping, application logic compiler 254 may retrieve the lines of code corresponding to logic 248, set parameters for the retrieved lines of codes, and arrange the lines of code 258. Application logic compiler 254 may also perform rearrangement or replacement of lines of code to increase the efficiency of executing the healthcare protocol by medical stations 140.

Content storage 270 stores various visual and/or audio content that may be linked to code 258. This content is presented to the patients or visitors of medical stations 140 when application 280 is executed on medical stations 140 to facilitate their understanding of diagnosis, treatment, actions or recommendations according to the healthcare protocols and invoked functionalities of medical stations 140. Content generator 274, on the other hand, generates visual and/or audio content 272 that is not pre-stored in content storage 270. Content generator 274 may include, among others, a 3D graphics engine (e.g., Unity available from Unity Technologies of San Francisco, California, and Unreal Engine of Epic Games available from Epic Games of Cary, North Carolina) and a speech synthesizer. Content generator 274 may also include generative artificial intelligence (AI) to create content to be presented at medical stations 140 in addition to or in place of content generated by the 3D graphics engine. Depending on code 258, relevant visual and/or audio content 272 may be generated by content generator 274 and provided to application packager 268.

Application packager 268 receives code 258 and content 272, links content 272 to the code, and organizes them to generate application 280 executable on medical stations 140. In one or more embodiments, audio content 272 is processed and sent separately from application 280, which is a compiled version of code 258. Application 280 (with or without content) may be sent to backend server 120 or medical stations 140 via network interface 206 for deployment.

FIG. 3A is a block diagram illustrating components of backend server 120, according to one embodiment. Backend server 120 may include, among other components, processor 302, memory 310, network interface 306 and bus 350 connecting these components. Backend server 120 may include other components not illustrated in FIG. 3A.

Processor 302 reads and executes instructions stored in memory 310 to perform various operations on backend server 120. Although only a single processor 302 is illustrated in FIG. 3A, multiple processors may be included in backend server 120. Processor 302 may be general-purpose or an embedded processor using any of a variety of instruction set architectures (ISAs), such as the x86, PowerPC, SPARC, RISC, ARM or MIPS ISAs, or any other suitable ISAs.

Network interface 306 is hardware or hardware in combination with firmware or software for communicating with medical stations 140 and/or protocol management device 130. For this purpose, network interface 406 may implement various wired or wireless protocols.

Memory 310 is a non-transitory storage medium for storing software modules. Memory 310 may include, among other software modules, application repository 312, patient information module 320, medical station management module 330 and treatment assessment module 340. Memory 310 may store other software modules not illustrated in FIG. 3A. Two or more software modules in FIG. 3A may be combined into a single module or a software module in FIG. 3A may be split up into multiple software modules. Memory 310 may take the form of any type of memory structure including, for example, dynamic random access memory (DRAM), synchronous DRAM (SDRAM), double data rate (DDR, DDR2, DDR3, etc.) RAMBUS DRAM (RDRAM), static RAM (SRAM) or a combination thereof.

Application repository 312 stores applications 280 to be sent to medical stations 140 for installation. Application repository 312 may retain the most recent versions of the applications 280 and may archive older versions of the applications 280. Newer applications or updated versions of the applications 280 may be automatically received from protocol management device 130 and be stored in application repository 312. In addition or alternatively, applications 416 may be transferred and stored in application repository 312 manually by human operators. Application repository 312 may also store metadata on applications 280, including but not limited to, related applications, stored versions of applications, ID of developers who developed the applications, and eligible membership to access the applications.

In other embodiments, backend server 120 does not include application repository 312. Rather, medical stations 140 receive applications directly from developers via network or through manual installation.

Patient information module 320 is a database including the list of patients and their membership information (e.g., name, age, gender, membership level, insurance information). Patient information may also include EMR of the patients that may be updated based on diagnostic operations performed at medical stations 140. Likewise, EMR or other patient information can be updated via a mobile device, via traditional medical facility visits, via a web interface, via a health app, or via any other suitable avenue. Patient information may be accessed by medical stations 140 to determine whether a patient should be granted access to medical stations 140 and/or certain applications 280 executable on medical stations 140.

Medical station management module 330 is a software module that performs various management operations related to medical stations 140. Medical station management module 330 may keep track of inventories of medical tools available in a medical station 140 and take actions to replenish them if inventories are running low. For this purpose, medical station management module 330 may send orders to warehouses to ship tools 448 to sites where medical stations 140 are located. Also, medical station management module 330 may instruct maintenance personnel to visit and carry out predetermined maintenance operations on a certain medical station.

Treatment assessment module 340 performs analysis to determine efficacy of treatments performed on patients. For this purpose, treatment assessment module 340 may receive patients' information from one or more medical stations 140. Various statistical analyses may be performed on the patients' information to determine efficacy or performance of treatment options. In some embodiments, the distribution of multiple medical stations 140 as described herein allows users to access care more frequently than having to visit traditional doctor's offices, allowing for a greater collection of patient data. Likewise, the medical stations 140 described herein enable more and different types of patient data to be collected. The treatment assessment module 340 can leverage this data to evaluate adherence and effectiveness of particular treatment options, which in turn can be leveraged to evaluate best courses of action or treatment to recommend to other users. The information of the patients or visitors may be provided in real-time to the treatment assessment module 340 to enable health service providers to evaluate, assess and choose treatment options. In addition, the information of the patients or visitors (such as demographic or geographic information, health risks, existing conditions, history, and the like) may be leveraged to recommend certain health apps or programs offered by the medical stations 140.

FIG. 3B is a block diagram of language model 154 in language module server 150, according to one embodiment. Language model 154 may be a large language model, and may be embodied using, among others architecture, an encoder-decoder transformer architecture. The transformer may include, among other components, an input embedding module 352, a first positional embedding module 354, a stack of encoders 356A through 356N (hereinafter collectively referred to as “encoders 356” or individually as “encoder 356”), an output embedding module 374, a second positional embedding module 378, and a stack of decoders 358A through 358N (hereinafter collectively referred to as “decoders 358” or individually as “decoder 358”). Outputs from the stack of encoders 356 are fed as inputs to the stack of decoders 358.

Input embedding module 352 receives input 351 and converts it into a set of contextual embeddings (also referred to herein as “input embeddings”). For this purpose, embedding module 412 splits input 351 into tokens. First positional embedding module 354 adds positional embeddings indicating the positions of each of the tokens. The token embeddings, the positional embeddings and other embeddings (if any) are concatenated into the contextual embeddings, and provided to the stack of encoders 356.

Each of the encoders 356 includes, among other components, a first multi-head attention module 362, a first add and normalize module 364, a first feed forward module 368, and a second add and normalize module 372. First multi-head attention module 362 uses multiple sets, or attention heads where each of the attention heads processes different aspects of context. For each token in input, attention scores for all other tokens are independently calculated by these attention heads. The computed attention weights from these different attention heads are then combined to create a comprehensive contextual representation for each token. First multi-head attention module 362 may be embodied using a neural network. The first add and normalize module 364 adds the input of a layer back to its output to maintain the flow of gradients during backpropagation and also performs layer normalization of the summed output for each input across the features. The output from the first add and normalize module 364 is fed to first feed forward module 368 and the second add and normalize module 372.

First feed forward module 368 combines the output of first multi-head attention module 362 with the input embeddings, and layer normalization is applied to standardize the activations within the layer. First feed forward module 368 may be embodied as a neural network. First feed forward module 368 may further include linear transformations followed by a nonlinear activation function (e.g., ReLU).

The output of the first feed forward module 368 is then provided to second add and normalize module 372 of decoders 358. Second add and normalize module 372 adds the output of first feed forward module 368 to the original input embeddings. Then the layer normalization is applied to standardize the activations within each layer. The output from the second add and normalize module 372 is then fed to the next encoder in the stack. The process of feeding the output from a previous encoder as an input to the subsequent encoder is repeated until the last encoder is reached. The output from the last encoder is then provided as the output of the stack of encoders 356 to decoders 358.

Decoder 358 generates output sequences based on the output provided by the stack of encoders 356. Decoder 358 may include, among other components, a masked multi-head attention module 382, a third add and normalize module 384, a second multi-head attention module 365, a fourth add and normalize module 388, a second feed forward module 390 and a fifth add and normalize module 392. Masked multi-head attention module 382 uses a masking technique to block or mask the attention to future tokens and enables only previous tokens to be attended to. The functions of third add and normalize module 384 are substantially identical to those of first add and normalize module 364 except for the difference in their inputs. Further, the functions of fourth add and normalize module 388 and fifth add and normalize module 392 of decoder 358 are substantially identical to those of second add and normalize module 372 of encoder 356 except for the difference in their inputs. Second multi-head attention module 386 and second feed forward module 390 perform substantially the same functions as those of first multi-head attention module 362 and first feed forward module 368, respectively, except for the differences in their inputs.

The output from fifth add and normalize module 392 is then fed to the next decoder in the stack. The process of feeding the output from a previous decoder as an input to the subsequent decoder is repeated until the last decoder is reached. The output from the last decoder is then provided as the output of language model 154. The output of the stack of encoders 356 is also fed back to output embedding module 374. Output embedding module 374 converts each token in the output of the decoder stack into a continuous vector representation in a manner substantially identical to input embedding module 352. Second positional embedding module 378 adds positional embeddings indicating the positions of each of the tokens in vector representation generated by output embedding module 374. The token embeddings, the positional embeddings and other embeddings (if any) are concatenated into the contextual embeddings, and provided to the stack of decoders 358. The process of processing at the decoders 358 are repeated as outputs from the stack of encoders 356 are received.

Although an encoder-decoder transformer architecture was used as language model 154 in the example of FIG. 3B, various other architectures may be used to embody language model 154. Language model 154 may be embodied, for example, using transformer-based architecture such as BERT, ALBERT, BART, BigBird, CamemBERT, ConvBERT, Data2VecText, DeBERTa, DeBERTa-v2, DistilBERT, ELECTRA, ERNIE, ESM, FlauBERT, FNet, Funnel Transformer, I-BERT, LayoutLM, Longformer, LUKE, mBART, MarianMT, MEGA, Megatron-BERT, MobileBERT, MPNet, MRA, MVP, Nezha, Nyströmformer, Perceiver, QDQBert, Reformer, RemBERT, ROBERTa, ROBERTa-PreLayerNorm, RoCBert, RoFormer, SqueezeBERT, TAPAS, Wav2Vec2, XLM, XLM-ROBERTa, XLM-ROBERTa-XL, X-MOD, and YOSO or may use other types of architecture such as mixture-of-experts. Language model 154 may also include multiple language models that are cascaded.

FIG. 4A is a block diagram of medical station 140, according to one embodiment. Medical station 140 may include, among other components, computing device 420, one or more display devices 430, input device 432 for receiving user input, drawer assemblies 440, sensors 450, network interface 460 and plug-in components 470. Medical station 140 may include other components not illustrated in FIG. 4A such as a turntable on which a patient stands for scanning of the patient's body with a body scanner, interfaces that enable third-party or modular equipment or sensors to couple with and provide data/power to and from the equipment, and any other suitable equipment that extends the functionality of the medical station 140.

Computing device 420 executes logic to interface and control other components of medical station 140. For this purpose, computing device 420 may include, among other components, processor 422, component interface 424, and memory 426. Processor 422 is a hardware circuit or hardware circuit in combination with firmware that executes instructions stored in memory to perform various functions on computing device 420. Memory 426 is a non-transitory storage medium for storing software modules executable on processor 422. Component interface 424 is hardware or hardware in combination with software that enables interfacing with medical tools 448 (as applicable), sensors 450, and/or plug-in components 470. Component interface 424 may include multiple subunits that enable computing device 420 to communicate with different components of medical station 140 over different protocols.

Display device 430 receives display signals from computing device 420 to display various images to a patient or a human assistant. In one embodiment, one of display devices 430 may be a touch screen that is embedded onto a wall of medical station 140. Multiple display devices may also be included in medical station 140. For example, a display device may be installed at an entry of medical station 140 to display information related to access to medical station 140 and/or types of medical services provided by medical station 140 while another device installed in the interior of medical station 140 may display graphical user interfaces to select and use a particular health application.

Input device 432 is a device for receiving user inputs. Input device 432 may be embodied as part of a display device (e.g., a touch sensor) or a separate component (e.g., a microphone, a mouse or a keyboard). More than one input device may be included in medical station 140 to support different types of user inputs.

Drawer assemblies 440 are mechanically operated drawers that enable a patient or a human assistant to selectively access a medical tool or medical supplies. A drawer assembly may be locked or opened through operation of actuator 444 that operates according to a control signal from computing device 420. Medical tools 448 may include, among others, wireless heart rate monitor, digital stethoscope, contactless thermometer, pap smear, blood draw kit, vaccine supplies, ear lavage, dermatoscope, or any other suitable device or equipment for self-administration or administration by a healthcare provider. At least some of these medical tools 448 communicate via component interface 424 to enable collection of patient information in real-time while other medical tools 448 are collected for further testing and analysis. In some embodiments, the drawer assemblies 440 provide an API, allowing third-party entities to interface with the medical station 140 by, for example, specifying drawer requirements (such as a size, temperature control, power requirements, and the like) needed for providing medicine, treatments, or testing equipment to an individual.

Sensors 450 are provided in medical station 140 to detect physical characteristics or pose of the patients. In one embodiment, sensors 450 may be embodied as a body scanner that detects dimensions of various parts of the patient's body. The body scanner may be used in conjunction with a turntable (not shown) that rotates the patient while the scanning is being performed. A glucose meter may also be provided in medical station 140 as one of sensors 450 to detect the patient's blood level with or without drawing blood from the patient. Additional sensors 450 can include a thermography sensor, a blood pressure measurement system, a blood oxygenation detection system, a heart rate monitor, a body position sensor, a glucose meter, or any other suitable sensor or measurement/detection system.

Network interface 460 is hardware or hardware in combination with firmware that enable medical station 140 to communicate with backend server 120 or protocol management device 130 via network 110. Network interface 460 may, for example, include antenna and circuits for communicating over the Internet via wireless communication and/or a network card for communicating via wired communication (e.g., Ethernet) or for communication via private networks or peer-to-peer networks.

Plug-in components 470 are physical components added to medical station 140 to expand functionality to medical station 140. Plug-in components 470 may include a digital stethoscope, a digital dermatoscope, a Pap smear kit, an EKG machine, an ultrasound device, a spirometer, a retinoscope, a blood draw kit, treatment kits (such as a cryo-gun, a ear lavage kit and the like), exercise materials (such as resistance bands), food and drinks, merchandise, prescription medicine, over-the-counter medicine, educational or informative materials, and the like. Functionalities of the medical station 140 that may leverage plug-in components 470 include but are not limited to: measuring blood oxygenation, measuring/listening to heart sounds, performing venipuncture, measuring weight or height, administering vaccines or other injections, performing a nasal swab, collecting a urine sample, measuring blood pressure, observing insides of a patient's ears/nose/mouth/throat, capturing images of a patient's skin, performing an ultrasound/x-ray/MRI on a patient, observing a patient's eyes (e.g., using an ophthalmoscope), performing an EKG, performing a CT scan, performing spirometry functions, analyzing a patient's posture, performing cardiac telemetry, performing tonometry functions, capturing retinal or other images of the patient's eyes or other organs/body parts, capturing thermal or hyperspectral images of the patient, and analyzing a patient for chemicals or other compounds.

Memory 426 stores health applications 280 that are software modules for interacting with the patients or visitors to provide medical or health related services to the patients or visitors. Each application 280 may be focused on different aspects of the medical/health services (e.g., a mental health checkup service, and a cardiovascular disease treatment service). In some embodiments, a different suite of applications developed and managed by different health care entities may be stored in memory 426. At least some of these applications 280 are developed automatically by protocol management device 130.

Some of the applications 280 may make one or more tools 448 in drawer assemblies 440 available to the patients during their operations. For this purpose, the applications 280 may cause component interface 424 of computing device 420 to timely send activation signals to appropriate drawer assemblies 440 while the applications 280 are active. If multiple medical tools 448 are to be accessed by a patient, the active application may send a series of activation signals to activate actuators 444 of relevant drawer assemblies 240 so that relevant medical tools 448 may be sequentially accessed by the patient.

Applications 280 may also operate plug-in components 470 to perform further tests on the patient or the visitor. The use or access to certain plug-in components 470 may be restricted to a subset of applications. For example, a plug-in component specifically designed for a medical service provider may be accessed or used only by an application developed or affiliated with the same medical service provider. By using plug-in components, an application 280 may expand the services it is capable of providing.

FIG. 4B is a block diagram illustrating user device 134, according to one embodiment. User device 134 may include, among other components, processor 484, memory 480, network interface 486 and bus 490 connecting these components. User device 134 may include other components not illustrated in FIG. 4A. Processor 484 reads and executes instructions stored in memory 480 to perform various operations, including launching and execution of health app 492. Network interface 486 is hardware or hardware in combination with firmware or software for communicating with medical stations 140 and/or backend server 120. For this purpose, network interface 406 may implement various wired or wireless protocols. Memory 480 is a non-transitory storage medium for storing software modules. Memory 310 may include, among other software modules, health app 492. Health app 492 enables a user of user device 134 to perform various actions associated with healthcare protocols such as providing user inputs on treatment, diagnosis or preventative actions, reserving a medical station, receiving notifications from backend server 120 and/or the medical station. In some embodiments, the healthcare protocols described herein are utilized through the user devices without using the medical stations. For example, all data and information relevant to the healthcare protocols may be received from the user devices 134 and/or backend server 120.

FIG. 5 is a conceptual diagram of a healthcare protocol and its mapping to functionalities of medical stations 140, according to one embodiment. Intermediate data 230 may be represented as a binary decision tree 510 or Boolean logic. Binary decision tree 510 includes plurality of nodes from which branches bifurcate. One or more of the nodes may be mapped to a certain functionality of medical stations 140.

Taking an example of binary decision tree 510 representing a cardiovascular healthcare protocol, first node 532 is associated with the systolic blood pressure of the patient or the visitor while a subsequent second node 534 is associated with diastolic blood pressure of the patient or the visitor, and third node 538 is associated with whether the patient or visitor has diabetes or kidney diseases. One or more nodes in binary decision tree 510 may be assigned to a segment (e.g., segment 520A, segment 520B, . . . , segment 520Z). Each segment 520 refers to one or more nodes of the intermediate data 230 that may be tested or performed by invoking a single unit of functionality at medical stations 140. In the example of FIG. 5, determination for segment 520A may be made by using the functionality of blood pressure module 522A of medical stations 140, determination of segment 520B may be made using the functionality of questionnaire module 522B of medical stations 140, and determination of segment 520C may be made by using blood sample module 522C of medical stations 140.

Functionalities 522 of medical stations may be categorized into different modules to facilitate the mapping operation. Such functionalities and categorization may be available from medical station information 244. In the example of FIG. 5, only three functionalities 522 of blood pressure module 522A, questionnaire module 522B and blood sample module 522C are illustrated for the sake of simplicity, but medical stations 140 in practice may implement many more modules assigned with their functionalities. Blood pressure module 522A in the example is a combination of hardware including blood pressure monitor and software for operating a drawer in medical stations 140 to present the blood pressure monitor to the patient or visitor with associated content. Questionnaire module 522B is software that presents various questions on one or more display devices 430 of medical station 140 and receives answers from the patient or visitor using input device 432 of medical station 140. In the example, questionnaire module 522B may be invoked to ask the patient or visitor whether he or she has diabetes or kidney disease, and receives an answer from the patient or visitor. Blood sample module 522C is a set of (i) hardware including as a blood sampling device and/or a blood analyzer, (ii) software that calls a human assistant to sample blood, and (iii) a human assistant to use the blood sampling device and/or the blood analyzer. Although FIG. 5 illustrates mapping only one segment to each of the different modules, multiple different segments of intermediate data 230 may be mapped to the same module.

After making determinations at relevant segments (e.g., 520A through 520Z), one or more actions (action 1 through N) are determined. For example, action 1 may indicate prescribing a type of medication, action 2 may indicate scheduling a follow-up appointment, action 3 may indicate dispensing of a mobile blood pressure monitor to the patient or visitor for continued use, etc. Although not illustrated in FIG. 5, some of the actions may provide a loop back to one or more segments 520A through 520Z for a subsequent round of making determinations. For example, action 4 may request a user to perform a number of sit-ups, and then return to segment 520A and repeat the subsequent segments to arrive at action 1 of prescribing a medication.

Mapping engine 238 generates logic 248 based on the mapping, as explained above with reference to FIG. 2. Specifically, mapping engine 238 maps segments in intermediate data 230 (e.g., binary decision tree 510 or Boolean logic) to functionalities/modules 522 of medical stations 140, chains the mapped functionalities/modules 522, and adds module-specific information (e.g., specific question to ask at questionnaire module 522B) to generate logic 248. Mapping engine 238 may also process intermediate data 230 into a semantic ontology of medical terms relating to, for example, diseases, medications and treatments (e.g., SNOMED CT).

Although the example of FIG. 5 uses a binary decision tree as intermediate data 230, the intermediate data 230 may be represented in different types of data such as a set of rules expressed using a predefined grammar (like a programming language). Further, the unit of functionalities of medical stations 140 may be more or less granular than what are presented in FIG. 5. For example, blood sample module 522C may be split further into a first module that tests only the level of cholesterol, and another module that tests only the level of triglycerides.

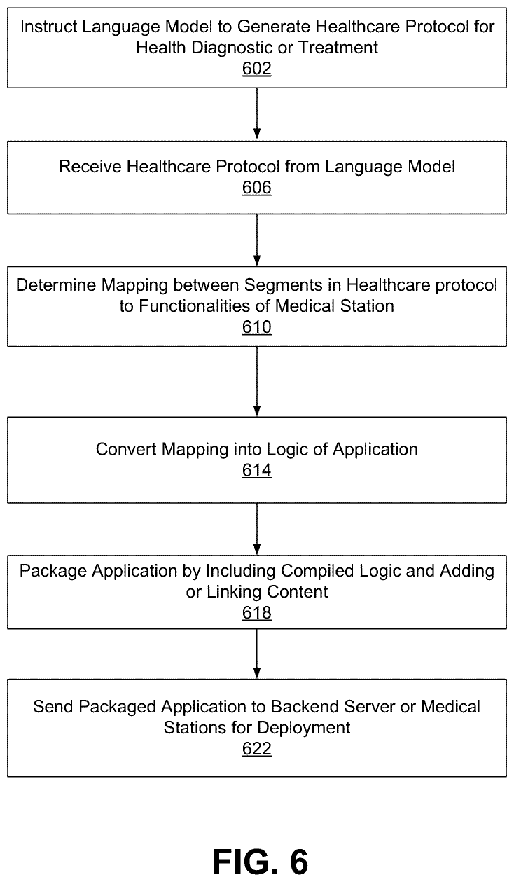

FIG. 6 is a flowchart illustrating operations at protocol management device 130, according to one embodiment. First, healthcare protocol analyzer 222 receives user input on a topic or field of healthcare, and instructs 602 an AI language model of language model server 150 to generate a healthcare protocol for diagnosis or treatment of users or prevent health issues of the users. The instruction may also cause collector 226 to gather materials on the corresponding topic or field of healthcare, and forward the gathered information to the AI language model.

In response, healthcare protocol analyzer 222 receives a healthcare protocol from language model server 150. Healthcare protocol analyzer 222 may convert the healthcare protocol into an intermediate format (e.g., a binary decision tree) that can be further processed.

Function mapper 234 determines 610 mapping between segments of the healthcare protocol to functionalities of a medical station. The mapping is then converted 614 into logic for an application. The logic may be a chain of sequences of the functionalities of the medical station. The logic may then be compiled into lines of code that may be executed on medical stations 140. In one or more embodiments, different versions of code may be compiled for medical stations of different configuration and capabilities.

The application is packaged 618 by including compiled logic in the form of code and adding/linking content associated with the logic. The content may be generated and stored in advance or generated upon demand using content generator 274.

The packaged application is sent 622 to backend server 120 or medical stations 140 for deployment. Backend server 120 may perform management operations (e.g., versioning and caching) on the received application, and propagate the application to medical stations 140. In some embodiments, backend server 120 may send the application to a subset of medical stations 140 or send different versions of the application to medical stations 140 of different configurations or capabilities.

The operations of FIG. 6 are merely illustrative. Additional processes may be added or some processes of FIG. 6 may be omitted. For example, a process of verifying the logic of the application may be added. Further, the sequence of processes in FIG. 6 may be modified. For example, the processes of determining 610 mapping and converting 614 the mapping may be performed in a single process or be performed in parallel.

While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure.

What is claimed is:

1. A method of generating an application for execution by a medical station, comprising:
sending instructions to a language model to generate a healthcare protocol, the healthcare protocol associated with diagnosis or treatment of a user or preventing health issues of the user accessing the medical station;
receiving the healthcare protocol from the language model in response to sending the instructions to the language model;
determining mapping between segments in the healthcare protocol and functionalities of the medical station, each of the segments including one or more nodes of the healthcare protocol;
generating logic corresponding to sequence of the segments and the mapping; and
generating an application incorporating the logic for execution by the medical station.

2. The method of claim 1, wherein the healthcare protocol is represented as a decision tree or a Boolean logic including the one or more nodes.

3. The method of claim 2, further comprising:
storing information on tools, sensors and plug-in components on the medical station, wherein the functionalities mapped to the segments are associated with the tools, sensors and plug-in components.

4. The method of claim 1, wherein the healthcare protocol indicates one or more actions to be taken by at least one of the user or the medical station according to determination at the segments.

5. The method of claim 1, further comprising:
collecting materials on the healthcare protocols from a plurality of sources;
processing the collected materials; and
feeding the processed materials to the language model to fine-tune or perform training of the language model.

6. The method of claim 5, wherein the collected material is processed and filtered according to one or more criteria.

7. The method of claim 1, further comprising generating content using at least one of a graphics engine, a speech synthesizer and a generative artificial intelligence, wherein the application further incorporates the generated content.

8. The method of claim 7, wherein generating the application comprises:
compiling the logic into code executable by the medical station;
linking the content to the executable code; and
packaging the executable code and the content into the application.

9. The method of claim 1, further comprising sending the application via a network for execution by the medical station.

10. A computing device for generating an application for execution by a medical station, comprising:
one or more processors; and
a memory storing instructions thereon, the instructions when executed by the one or more processors cause the one or more processors to:
send instructions to a language model to generate a healthcare protocol, the healthcare protocol associated with diagnosis or treatment of a user or preventing health issues of the user accessing the medical station;
receive the healthcare protocol from the language model responsive to sending the instructions to the language model;
determine mapping between segments in the healthcare protocol and functionalities of the medical station, each of the segments including one or more nodes of the healthcare protocol;
generate logic corresponding to sequence of the segments and the mapping; and
generate an application incorporating the logic for execution by the medical station.

11. The computing device of claim 10, wherein the healthcare protocol is represented as a decision tree or a Boolean logic including the one or more nodes.

12. The computing device of claim 11, wherein the instructions further cause the one or more processors to:
store information on tools, sensors and plug-in components on the medical station, wherein the functionalities mapped to the segments are associated with the tools, sensors and plug-in components.

13. The computing device of claim 10, wherein the healthcare protocol indicates one or more actions to be taken by at least one of the user or the medical station according to determination at the segments.

14. The computing device of claim 10, wherein the instructions further cause the one or more processors to:
collect materials on the healthcare protocols from a plurality of sources;
process the collected materials; and
feed the processed materials to the language model to fine-tune or perform training of the language model.

15. The computing device of claim 14, wherein the instructions to process the collected materials comprise instructions to filter the collected materials according to one or more criteria.

16. The computing device of claim 10, wherein the instructions further cause the one or more processors to generate content using at least one of a graphics engine, a speech synthesizer and a generative artificial intelligence, wherein the application further incorporates the generated content.

17. The computing device of claim 16, wherein the instructions to generate the application includes instructions to:
compile the logic into code executable by the medical station;
link the content to the executable code; and
package the executable code and the content into the application.

18. The computing device of claim 10, further comprising a network interface circuit configured to send the application to the medical station via a network.

19. A non-transitory storage medium storing instructions thereon, the instructions when executed by one or more processors cause the one or more processors to:
send instructions to a language model to generate a healthcare protocol, the healthcare protocol associated with diagnosis or treatment of a user or preventing health issues of the user accessing a medical station;
receive the healthcare protocol from the language model responsive to sending the instructions to the language model;
determine mapping between segments in the healthcare protocol and functionalities of the medical station, each of the segments including one or more nodes of the healthcare protocol;
generate logic corresponding to sequence of the segments and the mapping; and
generate an application incorporating the logic for execution by the medical station.

20. The non-transitory storage medium of claim 19, wherein the healthcare protocol is represented as a decision tree or a Boolean logic including the one or more nodes.
